SEQUENCE LISTING (2 sequences)

SEQ ID NO: 1 - RNA, 4284 nt, synthetic construct
  source 1..4284: mol_type = other RNA
gagaanaaacnagnanncnncnggnccccacagacncagagagaacccgccaccangnncgngnnccnggngcngcngccncnggngnccagccagngngngaaccngaccaccagaacacagcngccnccagccnacaccaacagcnnnaccagaggcgngnacnaccccgacaaggngnncaganccagcgngcngcacncnacccaggaccngnnccngccnnncnncagcaacgngaccnggnnccacgccanccacgngnccggcaccaanggcaccaagaganncgacaaccccgngcngcccnncaacgacggggngnacnnngccagcaccgagaagnccaacancancagaggcnggancnncggcaccacacnggacagcaagacccagagccngcngancgngaacaacgccaccaacgnggncancaaagngngcgagnnccagnncngcaacgaccccnnccngggcgncnacnaccacaagaacaacaagagcngganggaaagcgagnnccgggngnacagcagcgccaacaacngcaccnncgagnacgngncccagccnnnccnganggaccnggaaggcaagcagggcaacnncaagaaccngcgcgagnncgngnnnaagaacancgacggcnacnncaagancnacagcaagcacaccccnancaaccncgngcgggancngccncagggcnncncngcncnggaaccccnggnggancngcccancggcancaacancacccggnnncagacacngcnggcccngcacagaagcnaccngacaccnggcganagcagcagcgganggacagcnggngccgccgcnnacnangngggcnaccngcagccnagaaccnnccngcngaagnacaacgagaacggcaccancaccgacgccgngganngngcncngganccncngagcgagacaaagngcacccngaagnccnncaccgnggaaaagggcancnaccagaccagcaacnnccgggngcagcccaccgaanccancgngcggnnccccaanancaccaancngngccccnncggcgaggngnncaangccaccaganncgccncngngnacgccnggaaccggaagcggancagcaanngcgnggccgacnacnccgngcngnacaacnccgccagcnncagcaccnncaagngcnacggcgngnccccnaccaagcngaacgaccngngcnncacaaacgngnacgccgacagcnncgnganccggggagangaagngcggcaganngccccnggacagacaggcaagancgccgacnacaacnacaagcngcccgacgacnncaccggcngngnganngccnggaacagcaacaaccnggacnccaaagncggcggcaacnacaannaccngnaccggcngnnccggaagnccaancngaagcccnncgagcgggacancnccaccgagancnancaggccggcagcaccccnngnaacggcgnggaaggcnncaacngcnacnncccacngcagnccnacggcnnncagcccacaaanggcgngggcnancagcccnacagagnggnggngcngagcnncgaacngcngcangccccngccacagngngcggcccnaagaaaagcaccaancncgngaagaacaaangcgngaacnncaacnncaacggccngaccggcaccggcgngcngacagagagcaacaagaagnnccngccannccagcagnnnggccggganancgccganaccacagacgccgnnagaganccccagacacnggaaanccnggacancaccccnngcagcnncggcggagngncngngancaccccnggcaccaacaccagcaancaggnggcagngcngnaccaggacgngaacngnaccgaagngcccgnggccanncacgccgancagcngacaccnacanggcgggngnacnccaccggcagcaangngnnncagaccagagccggcngncngancggagccgagcacgngaacaanagcnacgagngcgacanccccancggcgcnggaancngcgccagcnaccagacacagacaaacagcccncggagagccagaagcgnggccagccagagcancanngccnacacaangncncngggcgccgagaacagcgnggccnacnccaacaacncnancgcnanccccaccaacnncaccancagcgngaccacagaganccngccngngnccangaccaagaccagcgnggacngcaccangnacancngcggcgannccaccgagngcnccaaccngcngcngcagnacggcagcnncngcacccagcngaanagagcccngacagggancgccgnggaacaggacaagaacacccaagaggngnncgcccaagngaagcagancnacaagaccccnccnancaaggacnncggcggcnncaannncagccaganncngcccganccnagcaagcccagcaagcggagcnncancgaggaccngcngnncaacaaagngacacnggccgacgccggcnncancaagcagnanggcganngncngggcgacanngccgccagggancngannngcgcccagaagnnnaacggacngacagngcngccnccncngcngaccgangagangancgcccagnacacancngcccngcnggccggcacaancacaagcggcnggacannnggagcaggcgccgcncngcaganccccnnngcnangcaganggccnaccggnncaacggcancggagngacccagaangngcngnacgagaaccagaagcngancgccaaccagnncaacagcgccancggcaaganccaggacagccngagcagcacagcaagcgcccngggaaagcngcaggacgnggncaaccagaangcccaggcacngaacacccnggncaagcagcngnccnccaacnncggcgccancagcncngngcngaacgananccngagcagacnggacccnccngaggccgaggngcagancgacagacngancacaggcagacngcagagccnccagacanacgngacccagcagcngancagagccgccgagannagagccncngccaancnggccgccaccaagangncngagngngngcngggccagagcaagagagnggacnnnngcggcaagggcnaccaccngangagcnncccncagncngccccncacggcgnggngnnncngcacgngacanangngcccgcncaagagaagaannncaccaccgcnccagccancngccacgacggcaaagcccacnnnccnagagaaggcgngnncgngnccaacggcacccannggnncgngacacagcggaacnncnacgagccccagancancaccaccgacaacaccnncgngncnggcaacngcgacgncgngancggcanngngaacaanaccgngnacgacccncngcagcccgagcnggacagcnncaaagaggaacnggacaagnacnnnaagaaccacacaagccccgacgnggaccngggcganancagcggaancaangccagcgncgngaacanccagaaagagancgaccggcngaacgaggnggccaagaancngaacgagagccngancgaccngcaagaacnggggaagnacgagcagnacancaagnggcccnggnacancnggcngggcnnnancgccggacnganngccancgnganggncacaancangcngngnngcangaccagcngcngnagcngccngaagggcngnngnagcngnggcagcngcngcaagnncgacgaggacganncngagcccgngcngaagggcgngaaacngcacnacacangangacncgagcnggnacngcangcacgcaangcnagcngccccnnncccgnccngggnaccccgagncncccccgaccncgggncccaggnangcncccaccnccaccngccccacncaccaccncngcnagnnccagacaccncccaagcacgcagcaangcagcncaaaacgcnnagccnagccacacccccacgggaaacagcagngannaaccnnnagcaanaaacgaaagnnnaacnaagcnanacnaaccccagggnnggncaannncgngccagccacacccnggagcnagcaaaaaaaaaaaaaaaaaaaaaaaaaaaaaagcanangacnaaaaaaaaaaaaaaaaaaaaaaaaaaaaaaaaaaaaaaaaaaaaaaaaaaaaaaaaaaaaaaaaaaaaaa

SEQ ID NO: 2 - RNA, 4284 nt, synthetic construct
  source 1..4284: mol_type = other RNA
gagaataaactagtattcttctggtccccacagactcagagagaacccgccaccatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2025/0134989 A1
(43) Pub. Date: May 1, 2025

(54) USE OF NOVEL POLYMER FOR PREVENTING SARS-COV-2

(71) Applicants: POSTECH RESEARCH AND BUSINESS DEVELOPMENT FOUNDATION, Pohang-si (KR); Korea National Institute of Health, Cheongju-si (KR)

(72) Inventors: Won Jong KIM, Pohang-si (KR); Hong Lyun KIM, Pohang-si (KR); So Hee PARK, Cheongju-si (KR); Soo-Min KIM, Cheongju-si (KR); Jung-Ah LEE, Cheongju-si (KR); Eunkyung SHIN, Cheongju-si (KR); You-Jin KIM, Cheongju-si (KR); Hye Sook JEONG, Cheongju-si (KR); Dokeun KIM, Cheongju-si (KR)

(73) Assignees: POSTECH RESEARCH AND BUSINESS DEVELOPMENT FOUNDATION, Pohang-si (KR); Korea National Instityte of Health, Cheongju-si (KR)

(21) Appl. No.: 18/931,284

(22) Filed: Oct. 30, 2024

(30) Foreign Application Priority Data
Oct. 31, 2023 (KR) 10-2023-0148620

(51) Int. Cl. A61K 39/215; A61K 9/51; A61K 39/00; A61K 45/06; A61P 31/14; C12N 7/00

(52) U.S. Cl. CPC A61K 39/215; A61K 9/5153; A61K 45/06; A61P 31/14; C12N 7/00; A61K 2039/53; A61K 2039/55555; C12N 2770/20034

(57) ABSTRACT

The present invention relates to a novel vaccine composition for coronavirus infection-19 comprising a polymer-based mRNA carrier. Specifically, the present invention provides a vaccine composition comprising an mRNA carrier using a novel polymer capable of delivering a negatively charged genetic material such as mRNA, and the vaccine composition has excellent effects of enhanced in vivo immune activity such as high antibody formation ability and increased interferon-gamma production.

### CROSS-REFERENCE TO RELATED APPLICATION

This application claims priority to Korean Patent Application No. 10-2023-0148620 filed Oct. 31, 2023, the entire disclosure of which is incorporated herein by reference.

### INCORPORATION BY REFERENCE OF SEQUENCE LISTING

The content of the electronically submitted sequence listing, file name: Q302606_sequence listing as filed; size: 11,514 bytes; and date of creation: Oct. 27, 2024, filed herewith, is incorporated herein by reference in its entirety.

### TECHNICAL FIELD

The present invention relates to a use of a novel polymer-based mRNA carrier for preventing SARS-COV2-2, and more specifically, to a polymer-based carrier having high COVID-19 immune activity by forming an mRNA carrier by mixing a poly beta-aminoester-based or poly beta-aminoacrylate-based polymer and mRNA.

### BACKGROUND ART

Severe acute respiratory syndrome coronavirus 2 (SARS-COV-2) is the causative virus of coronavirus infection 2019 (COVID-19; coronavirus disease 2019), which has been prevalent since 2020. It belongs to the beta-coronavirus genus and comprises spike glycoprotein, a class I viral fusion protein.

Unlike other coronaviruses, SARS-COV-2 causes severe infection of the lower respiratory tract and has a high mortality rate. The main symptoms of SARS-COV-2 infection include high fever, cough, sore throat, and muscle pain. In some patients, respiratory distress and a decrease in immune cells have even been reported. Due to the high mortality rate and transmission rate of SARS-COV-2, many countries have gone through lockdowns since 2020, and various drugs have been developed to prevent and treat SARS-COV-2 to reduce transmission and prevent severe cases.

COVID-19 vaccines are used to prevent SARS-COV-2 infection and prevent severe cases of COVID-19, and more than 80% of the entire population in South Korea has been vaccinated with COVID-19 vaccines. Ad26.COV2.S COVID-19 vaccine from Janssen, NVX-CoV2373 COVID-19 vaccine from Novavax, BNT162b2 COVID-19 vaccine from Pfizer, and mRNA-1273 COVID-19 vaccine from Moderna have been used internationally, but the most widely adopted vaccines in each country are COVID-19 vaccines from Pfizer and Moderna, and the two companies accounted for approximately 83% of COVID-19 vaccine sales in 2021. Their vaccines are based on mRNA, and have been widely adopted due to their advantages of being able to be developed in a short period of time and having high efficacy compared to existing vaccines.

mRNA is a substance that carries genetic information for producing proteins inside the human body. Through mRNA delivery, it is possible to induce the production of proteins that originally exist inside the human body, as well as the production of proteins that do not exist in the body. Since the outbreak of coronavirus-19, the development of mRNA carriers has been actively progressing as mRNA vaccines that activate immune responses through mRNA delivery have been utilized. Several lipid nanoparticle-based mRNA carriers have been developed, but they have problems due to rapid diffusion to organs other than the target site (off-target effect) and high immunogenicity, which can cause various side effects such as myocarditis and pericarditis. In addition, since vaccines are administered at most 2 to 4 times, the possibility of exposure to these side effects increases. Therefore, there is a need to develop an mRNA carrier that has high mRNA expression efficiency, low off-target effect, relatively low immunogenicity, and fewer side effects.

The present inventors studied mRNA delivery using polymers to improve the shortcomings of conventional mRNA carriers. As a result, they discovered a novel polymer with high in vivo mRNA delivery efficiency, and discovered that high in vivo immune activity was achieved when it was used with SARS-COV-2 protein mRNA. Based on the above, the present inventors completed the present invention.

### DISCLOSURE OF THE INVENTION

### Technical Problem

An object of the present invention is to provide a novel vaccine composition for coronavirus infection-19 comprising a novel polymer-based mRNA carrier with high in vivo immune activity.

### Solution to Problem

The present invention provides a vaccine composition for SARS-COV-2 comprising a SARS-COV-2 spike glycoprotein mRNA, and a polymer comprising a repeating unit of Formula 1 below.

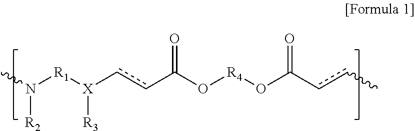

in the formula,
R1 is a bond or ethylene,
R2 and R3 are each independently (i) hydrogen, (ii) C2-5 alkyl that may be substituted with hydroxy, or (iii) N-methylpiperidine,
X is a nitrogen atom or a bond, and when X is a bond, said R3 is absent, and
R4 is a linear or methyl-substituted C3-12 alkylene, and 1 to 5 carbon atoms of said alkylene may be replaced with oxygen.

The repeating unit may be n (an integer from 1 to 500), and in this case, Formula 1 may be expressed as follows.

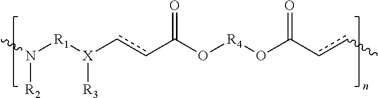

(The definitions of the groups are as defined in relation to Formula 1 above)

In the present invention, the SARS-COV-2 spike glycoprotein mRNA may be composed of a nucleic acid molecule represented by SEQ ID NO: 1.

In one embodiment, the vaccine composition may be characterized in that the polymer comprises a repeating unit selected from the group consisting of repeating units having structures set forth in the table below.

| Formula | Structure |
|---|---|
| 2 | 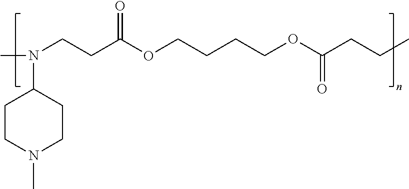 |
| 3 | 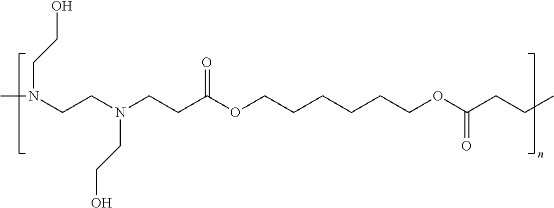 |
| 4 | 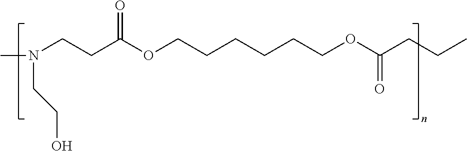 |
| 5 | 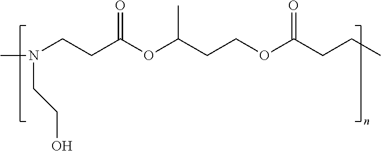 |
| 6 | 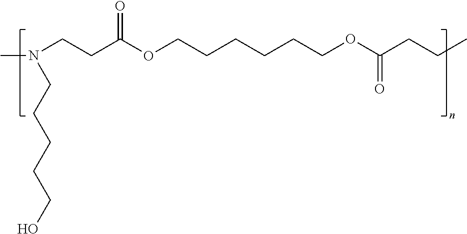 |
| 7 | 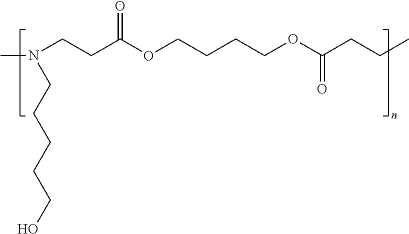 |
| 8 | 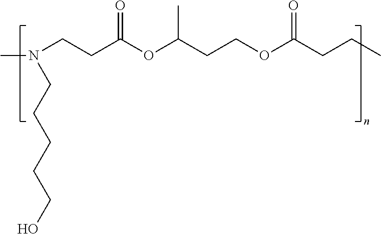 |
| 9 | 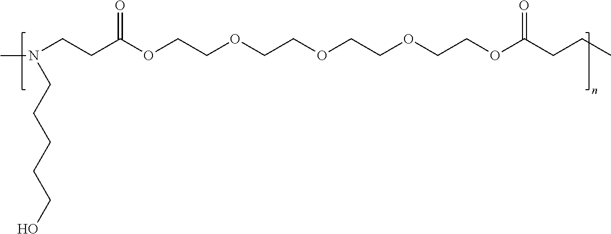 |
| 10 | 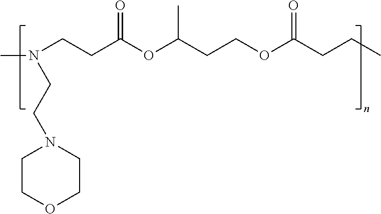 |

n as set forth in the table above is an integer from 1 to 500.

In one embodiment, the vaccine composition may be characterized in that the polymer has one structure selected from the group consisting of structures set forth in the table below.

| Formula | Structure |
|---|---|
| 8 | 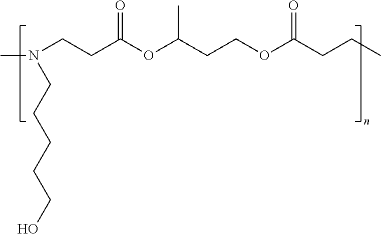 |
| 9 | 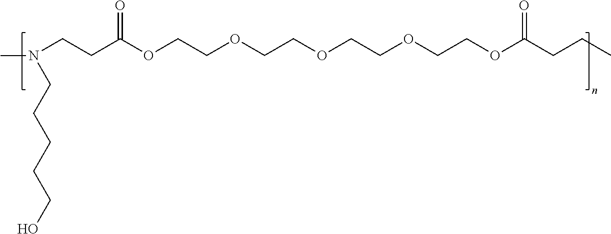 |
| 10 | 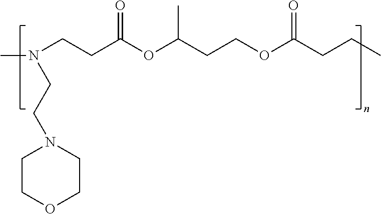 |

n as set forth in the table above is an integer from 1 to 500.

In one embodiment, the vaccine composition may be characterized in that the polymer comprises a repeating unit of Formula 8 below.

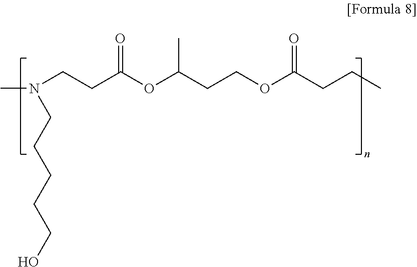

(in the formula, n is an integer from 1 to 500)

In one embodiment, the vaccine composition may be characterized in that the polymer comprises a repeating unit of Formula 9 below.

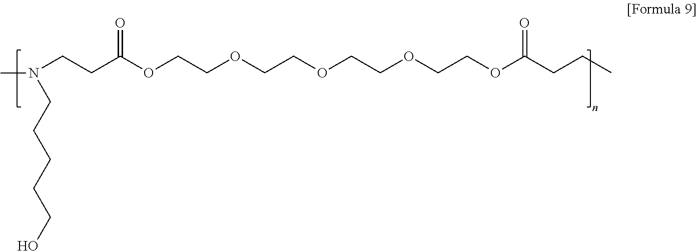

(in the formula, n is an integer from 1 to 500)

In one embodiment, the vaccine composition may be characterized in that the polymer comprises a repeating unit of Formula 10 below.

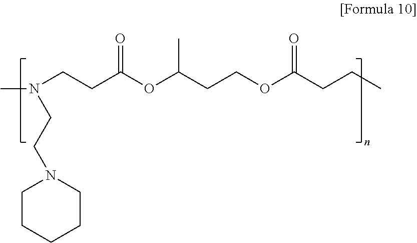

(in the formula, n is an integer from 1 to 500)

The vaccine composition according to the present invention preferably does not comprise lipid nanoparticles (LNPs).

In one embodiment, the vaccine composition may further comprise an immune enhancer.

In another embodiment, the presend disclosure provides a method of inducing immune response against SARS-COV-2 comprising: administering an effective amount of the vaccine composition according to the present disclosure to a subject in need thereof.

### Effects of Invention

The novel vaccine composition for coronavirus infection-19 comprising a polymer-based mRNA carrier according to the present invention has excellent effects of enhanced in vivo immune activity such as high antibody formation ability and increased interferon-gamma production.

### DETAILED DESCRIPTION FOR CARRYING OUT THE INVENTION

The preparation examples and examples of the present disclosure will be described in detail so that those of ordinary skill in the art to which the present invention belongs can easily practice the present invention. However, the present invention may be implemented in various forms and is not limited to the preparation examples and examples described herein.

Throughout the present specification, “polymer” and “high molecule” are used interchangeably with each other as having the same meaning.

In the present specification, the term “repeating unit” used to describe a “polymer” means a structure in which the same structure is repeated two or more times throughout the length of the polymer, and the polymer may not necessarily be composed solely of the repeating unit. For example, the polymer may further comprise branching structures and/or end capping agents in addition to the given repeating unit.

As used herein, the term “substituted”, for example, “substituted alkyl,” means that one or more hydrogen atoms of the alkyl are each independently replaced by a non-hydrogen substituent. The substituent may include, but is not limited to, any of the substituents described herein, for example, halogen, hydroxyl, alkyl, hydroxyalkyl, haloalkyl, cyanoalkyl, alkoxyalkyl, carbonyl (for example, carboxyl, alkoxycarbonyl, formyl, or acyl), thiocarbonyl (for example, thioester, thioacetate, or thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, aryl, or heteroaryl. The substituted moiety on the hydrocarbon chain may itself be substituted, if desired.

As used herein, an “alkyl group” is a hydrocarbon having substituted or unsubstituted primary, secondary, tertiary and/or quaternary carbon atoms, and includes a saturated aliphatic group which may be straight-chain, branched, cyclic, or a combination thereof. For example, the alkyl group may have 1 to 20 carbon atoms (i.e., C1-C20 alkyl), 1 to 10 carbon atoms (i.e., C1-C10 alkyl), or 1 to 6 carbon atoms (i.e., C1-C6 alkyl). Examples of suitable alkyl groups may include methyl (Me, —CH3), ethyl (Et, —CH2CH3), 1-propyl (n-Pr, n-propyl, —CH2CH2CH3), 2-propyl (i-Pr, i-propyl, —CH(CH3)2), 1-butyl (n-Bu, n-butyl, —CH2CH2CH2CH3), 2-methyl-1-propyl (i-Bu, i-butyl, —CH2CH(CH3)2), 2-butyl (s-Bu, s-butyl, —CH(CH3)CH2CH3), 2-methyl-2-propyl (t-Bu, t-butyl, —C(CH3)3), 1-pentyl (n-pentyl, —CH2CH2CH2CH2CH3), 2-pentyl (—CH(CH3)CH2CH2CH3), 3-pentyl (—CH(CH2CH3)2), 2-methyl-2-butyl (—C(CH3)2CH2CH3), 3-methyl-2-butyl (—CH(CH3)C (CH3)2), 3-methyl-1-butyl (—CH2CH2CH(CH3)2), 2-methyl-1-butyl (—CH2CH(CH3)CH2CH3), 1-hexyl (—CH2CH2CH2CH2CH2CH3), 2-hexyl (—CH(CH3)CH2CH2CH2CH3), 3-hexyl (—CH(CH2CH3)(CH2CH2CH3)), 2-methyl-2-pentyl (—C(CH3)2CH2CH2CH3), 3-methyl-2-pentyl (—CH(CH3)CH(CH3)CH2CH3), 4-methyl-2-pentyl (—CH(CH3)CH2CH(CH3)2), 3-methyl-3-pentyl (—C(CH3)(CH2CH3)2), 2-methyl-3-pentyl (—CH(CH2CH3)CH(CH3)2), 2,3-dimethyl-2-butyl (—C(CH3)2CH(CH3)2), 3,3-dimethyl-2-butyl (—CH (CH3)C(CH3)3), and octyl (—(CH2)7CH3), but are not limited thereto.

Furthermore, as used throughout the specification, examples, and claims, the term “alkyl” is intended to encompass both unsubstituted and substituted alkyl groups, the latter of which refers to an alkyl moiety having a substituent replacing a hydrogen on one or more carbon atoms of the hydrocarbon backbone, including haloalkyl groups such as trifluoromethyl.

As used herein, the term “alkylene” refers to a saturated hydrocarbon group having two valencies, which may be branched, straight-chain, cyclic, or a combination thereof, and which is derived by removing two hydrogen atoms from the same carbon atom or from two different carbon atoms of a parent alkane. For example, an alkylene group may have from 1 to 20 carbon atoms, from 1 to 10 carbon atoms, or from 1 to 6 carbon atoms. Examples of suitable alkylene groups may include, but are not limited to, methylene (—CH2—) and 1,2-ethylene (—CH2—CH2—).

As used herein, the term “acrylate monomer” refers to a monomer in the form of an ester derivative of acrylic acid, and may be selected from the group consisting of acrylate monomers set forth in Table 1 below.

TABLE 1

| No. | Structural formula |
|---|---|
| B | 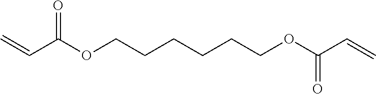 [SMILES: C=CC(=O)OCCCCCCOC(=O)C=C] |
| C | 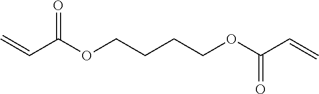 |
| D | 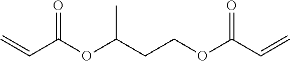 |
| E | 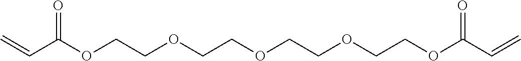 [SMILES: C=CC(=O)OCCOCCOCCOCCOC(=O)C=C] |

As used herein, the term “amine monomer” refers to a monomer comprising an amine functional group, and may be preferably selected from the group consisting of amine monomers set forth in Table 2 below.

TABLE 2

| No. | Structural formula |
|---|---|
| 4 | 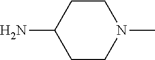 |
| 6 | 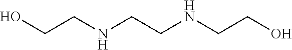 |
| 7 | 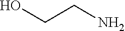 |
| 8 | 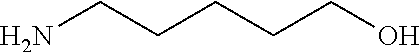 |
| 9 | 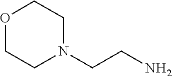 |

The vaccine composition of the present invention may be prepared in any suitable, pharmaceutically acceptable formulation. For example, it may be prepared in the form of a ready-to-use solution or suspension, a concentrated stock solution suitable for dilution prior to administration, or a reconstitutable form, such as a lyophilized, freeze-dried, or frozen formulation.

The vaccine composition of the present invention may be formulated including a pharmaceutically acceptable carrier. The pharmaceutically acceptable carrier typically includes a diluent, an excipient, a stabilizer, a preservative, and the like. For example, diluents that can be included in the vaccine composition of the present invention may include non-aqueous solvents such as propylene glycol, polyethylene glycol, vegetable oils such as olive oil and peanut oil, and aqueous solvents such as saline and water comprising a buffer medium; excipients may include starch, glucose, lactose, sucrose, gelatin, malt, rice, wheat flour, chalk, silica gel, sodium stearate, glycerol monostearate, talc, sodium chloride, anhydrous skim milk, glycerol, propylene, glycol, water, ethanol, and the like; and stabilizers may include carbohydrates such as sorbitol, mannitol, starch, sucrose, dextran, glutamate, and glucose; and proteins such as animal, vegetable or microbial proteins such as powdered milk, serum albumin, and casein. In addition, preservatives may include thimerosal, merthiolate, gentamicin, neomycin, nystatin, amphotericin B, tetracycline, penicillin, streptomycin, polymyxin B, and the like.

The vaccine composition of the present invention may further comprise an antigen adjuvant. The antigen adjuvant is one or more substances that enhance an immune response to an antigen, and may be, for example, one or more selected from the group consisting of complete Freund, incomplete Freund, saponin, gel-like aluminum adjuvants, surface-active substances (e.g., lysolecithin, pluronic glycol, polyanions, peptides, oil or hydrocarbon emulsions, etc.), vegetable oils (cottonseed oil, peanut oil, corn oil, etc.), and vitamin E acetate, but is not limited thereto.

The vaccine composition of the present invention may be administered parenterally or orally depending on the intended method, and the dosage range varies depending on the patient's weight, age, sex, health condition, diet, administration time, administration method, excretion rate, and the severity of the disease. In addition, the prophylactically or therapeutically effective amount of the composition may vary depending on the administration method, target site, and patient's condition, and when used in the human body, the dosage should be determined as an appropriate amount by considering both safety and effectiveness.

The vaccine composition may further comprise an immune enhancer. The additionally included immune enhancer may be at least one selected from the group consisting of aluminum salts, toll-like receptor (TLR) agonists, monophosphoryl lipid A (MLA), synthetic lipid A, lipid A mimetics or analogues, MLA derivatives, cytokines, saponins, muramyl dipeptide (MDP) derivatives, CpG oligos, lipopolysaccharides (LPS) of gram-negative bacteria, polyphosphazenes, emulsions, virosomes, cochleates, poly (lactide-co-glycolide) (PLG) microparticles, poloxamer particles, microparticles, liposomes, complete Freund's adjuvant (CFA), and incomplete Freund's adjuvant (IFA), but is not limited thereto.

As used herein, the term “prevention” refers to any action of inhibiting or delaying the onset of a disease by administration of a composition, and “treatment” refers to any action in which symptoms of a subject suspected of and suffering from a disease are improved or beneficially changed by administration of a composition.

Hereinafter, the present invention will be described in more detail through the examples, but the following examples are for illustrative purposes only and are not intended to limit the scope of the present invention.

### PREPARATION EXAMPLE 1

### Preparation of Polymer 4C

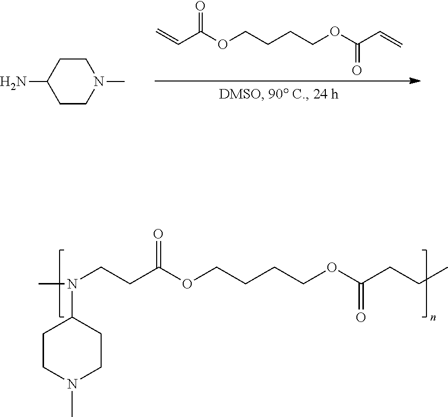

Acrylate monomer C (1000 mg) was placed in a 20 ml vial, and then amine monomer 4 was added to the vial so that the molar ratio with the acrylate monomer was 1.04. DMSO was added to the vial so that the final concentration was 1000 mg/ml. Thereafter, the reaction was performed at 90° C. for 24 hours to obtain Polymer 4C.

### PREPARATION EXAMPLE 2

### Preparation of Polymer 6B

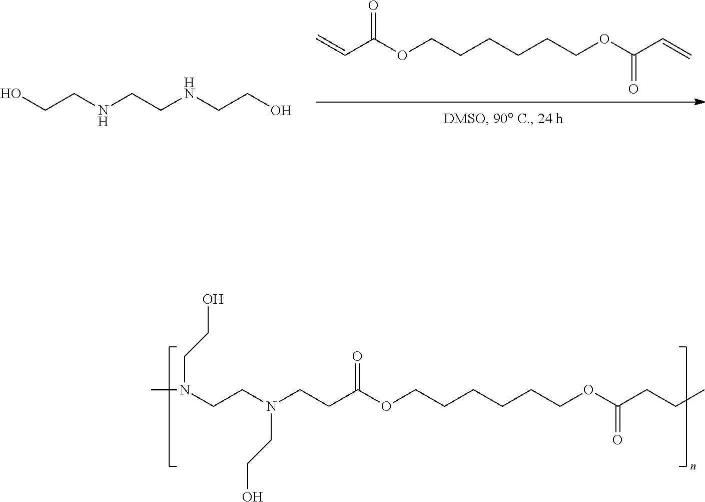

Acrylate monomer B (1000 mg) was placed in a 20 ml vial, and then amine monomer 6 was added to the vial so that the molar ratio with the acrylate monomer was 1.04. DMSO was added to the vial so that the final concentration was 1000 mg/ml. Thereafter, the reaction was performed at 90° C. for 24 hours to obtain Polymer 6B.

### PREPARATION EXAMPLE 3

### Preparation of Polymer 7B

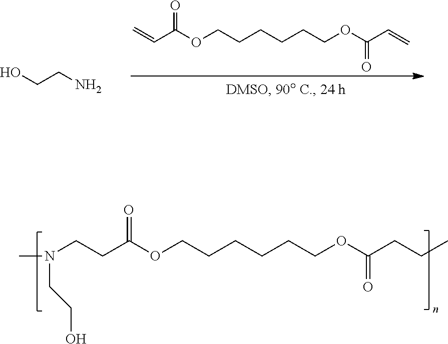

Acrylate monomer B (1000 mg) was placed in a 20 ml vial, and then amine monomer 7 was added to the vial so that the molar ratio with the acrylate monomer was 1.04. DMSO was added to the vial so that the final concentration was 1000 mg/ml. Thereafter, the reaction was performed at 90° C. for 24 hours to obtain Polymer 7B.

### PREPARATION EXAMPLE 4

### Preparation of Polymer 7D

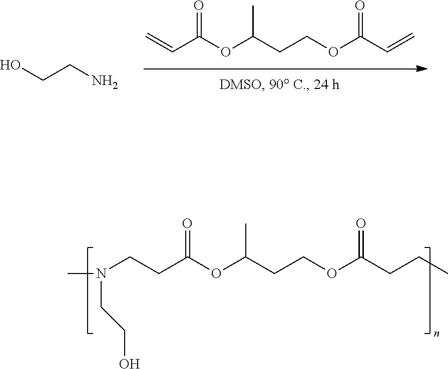

Acrylate monomer D (1000 mg) was placed in a 20 ml vial, and then amine monomer 7 was added to the vial so that the molar ratio with the acrylate monomer was 1.04. DMSO was added to the vial so that the final concentration was 1000 mg/ml. Thereafter, the reaction was performed at 90° C. for 24 hours to obtain Polymer 7D.

### PREPARATION EXAMPLE 5

### Preparation of Polymer 8B

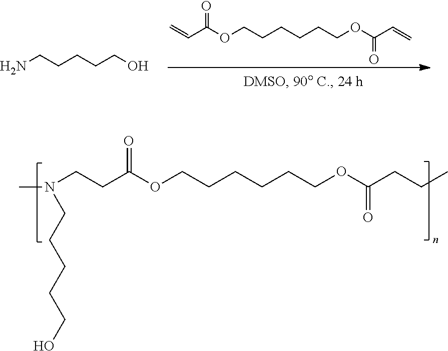

Acrylate monomer B (1000 mg) was placed in a 20 ml vial, and then amine monomer 8 was added to the vial so that the molar ratio with the acrylate monomer was 1.04. DMSO was added to the vial so that the final concentration was 1000 mg/ml. Thereafter, the reaction was performed at 90° C. for 24 hours to obtain Polymer 8B.

### PREPARATION EXAMPLE 6

### Preparation of Polymer 8C

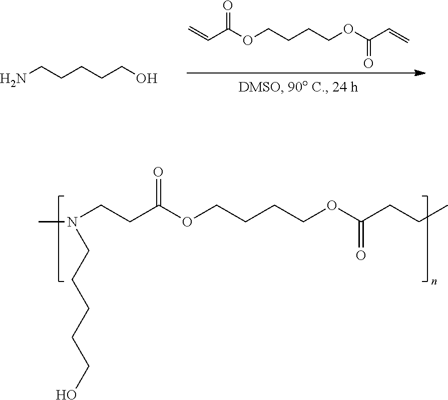

Acrylate monomer C (1000 mg) was placed in a 20 ml vial, and then amine monomer 8 was added to the vial so that the molar ratio with the acrylate monomer was 1.04. DMSO was added to the vial so that the final concentration was 1000 mg/ml. Thereafter, the reaction was performed at 90° C. for 24 hours to obtain Polymer 8C.

### PREPARATION EXAMPLE 7

### Preparation of Polymer 8D

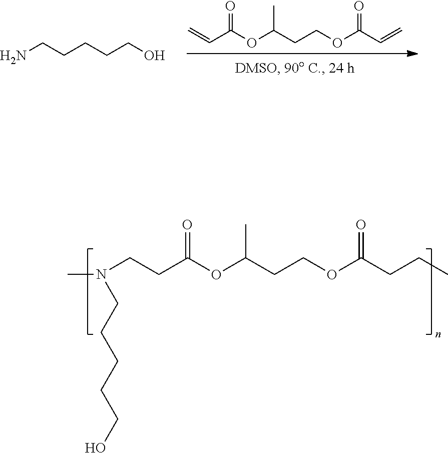

Acrylate monomer D (1000 mg) was placed in a 20 ml vial, and then amine monomer 8 was added to the vial so that the molar ratio with the acrylate monomer was 1.04. DMSO was added to the vial so that the final concentration was 1000 mg/ml. Thereafter, the reaction was performed at 90° C. for 24 hours to obtain Polymer 8D.

### PREPARATION EXAMPLE 8

### Preparation of Polymer 8E

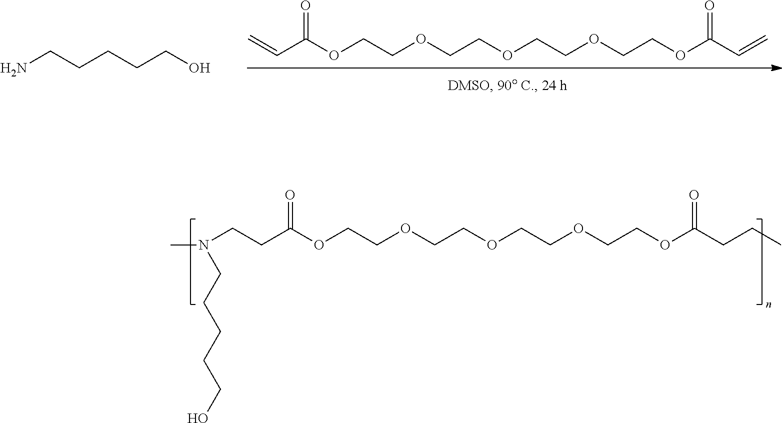

Acrylate monomer E (1000 mg) was placed in a 20 ml vial, and then amine monomer 8 was added to the vial so that the molar ratio with the acrylate monomer was 1.04. DMSO was added to the vial so that the final concentration was 2500 mg/ml. Thereafter, the reaction was performed at 90° C. for 24 hours to obtain Polymer 8E.

### PREPARATION EXAMPLE 9

### Preparation of Polymer 9D

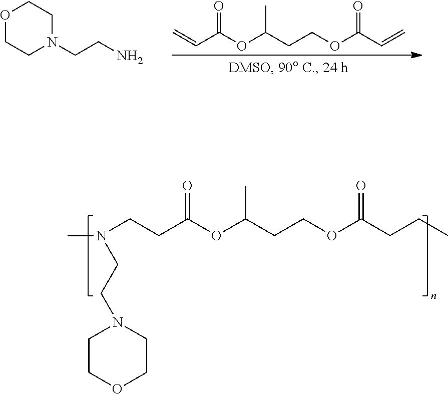

Acrylate monomer D (1000 mg) was placed in a 20 ml vial, and then amine monomer 9 was added to the vial so that the molar ratio with the acrylate monomer was 1.04. DMSO was added to the vial so that the final concentration was 1000 mg/ml. Thereafter, the reaction was performed at 90° C. for 24 hours to obtain Polymer 9D.

### EXAMPLE 1

### Preparation of Polymer Nanoparticle (PNP)

A polymer nanoparticle (PNP), which is a nucleic acid carrier of the present invention, was prepared by the following method by adding Covid-19 spike protein mRNA of SEQ ID NO: 1 to the polymer prepared by any one of Preparation Examples 1 to 9 above.

The polymer prepared by any one of Preparation Examples 1 to 9 above was diluted in a pH 5.0, 25 mM sodium acetate buffer at a ratio for each polymer. Thereafter, 5 μl of the polymer solution and 5 μl of mRNA at a concentration of 1 mg/ml were mixed and vortexed lightly. Thereafter, it was stabilized at 4° C. for 30 minutes, and 40 μl of PBS was added to make a final volume of 50 μl.

TABLE 3

| SEQ ID NO | RNA sequence |
|---|---|
| 1 | GAGAANAAACNAGNANNCNNCNGGNCCCCACAGACNCAGAGAGAACCCGCCACCA |
| | NGNNCGNGNNCCNGGNGCNGCNGCCNCNGGNGNCCAGCCAGNGNGNGAACCNGAC |
| | CACCAGAACACAGCNGCCNCCAGCCNACACCAACAGCNNNACCAGAGGCGNGNACN |
| | ACCCCGACAAGGNGNNCAGANCCAGCGNGCNGCACNCNACCCAGGACCNGNNCCNG |
| | CCNNNCNNCAGCAACGNGACCNGGNNCCACGCCANCCACGNGNCCGGCACCAANGG |
| | CACCAAGAGANNCGACAACCCCGNGCNGCCCNNCAACGACGGGGNGNACNNNGCCA |
| | GCACCGAGAAGNCCAACANCANCAGAGGCNGGANCNNCGGCACCACACNGGACAGC |
| | AAGACCCAGAGCCNGCNGANCGNGAACAACGCCACCAACGNGGNCANCAAAGNGN |
| | GCGAGNNCCAGNNCNGCAACGACCCCNNCCNGGGCGNCNACNACCACAAGAACAAC |
| | AAGAGCNGGANGGAAAGCGAGNNCCGGGNGNACAGCAGCGCCAACAACNGCACCN |
| | NCGAGNACGNGNCCCAGCCNNNCCNGANGGACCNGGAAGGCAAGCAGGGCAACNN |
| | CAAGAACCNGCGCGAGNNCGNGNNNAAGAACANCGACGGCNACNNCAAGANCNAC |
| | AGCAAGCACACCCCNANCAACCNCGNGCGGGANCNGCCNCAGGGCNNCNCNGCNCN |
| | GGAACCCCNGGNGGANCNGCCCANCGGCANCAACANCACCCGGNNNCAGACACNGC |
| | NGGCCCNGCACAGAAGCNACCNGACACCNGGCGANAGCAGCAGCGGANGGACAGC |
| | NGGNGCCGCCGCNNACNANGNGGGCNACCNGCAGCCNAGAACCNNCCNGCNGAAG |
| | NACAACGAGAACGGCACCANCACCGACGCCGNGGANNGNGCNCNGGANCCNCNGA |
| | GCGAGACAAAGNGCACCCNGAAGNCCNNCACCGNGGAAAAGGGCANCNACCAGAC |
| | CAGCAACNNCCGGGNGCAGCCCACCGAANCCANCGNGCGGNNCCCCAANANCACCA |
| | ANCNGNGCCCCNNCGGCGAGGNGNNCAANGCCACCAGANNCGCCNCNGNGNACGCC |
| | NGGAACCGGAAGCGGANCAGCAANNGCGNGGCCGACNACNCCGNGCNGNACAACN |
| | CCGCCAGCNNCAGCACCNNCAAGNGCNACGGCGNGNCCCCNACCAAGCNGAACGAC |
| | CNGNGCNNCACAAACGNGNACGCCGACAGCNNCGNGANCCGGGGAGANGAAGNGC |
| | GGCAGANNGCCCCNGGACAGACAGGCAAGANCGCCGACNACAACNACAAGCNGCCC |
| | GACGACNNCACCGGCNGNGNGANNGCCNGGAACAGCAACAACCNGGACNCCAAAG |
| | NCGGCGGCAACNACAANNACCNGNACCGGCNGNNCCGGAAGNCCAANCNGAAGCCC |
| | NNCGAGCGGGACANCNCCACCGAGANCNANCAGGCCGGCAGCACCCCNNGNAACGG |
| | CGNGGAAGGCNNCAACNGCNACNNCCCACNGCAGNCCNACGGCNNNCAGCCCACAA |
| | ANGGCGNGGGCNANCAGCCCNACAGAGNGGNGGNGCNGAGCNNCGAACNGCNGCA |
| | NGCCCCNGCCACAGNGNGCGGCCCNAAGAAAAGCACCAANCNCGNGAAGAACAAA |
| | NGCGNGAACNNCAACNNCAACGGCCNGACCGGCACCGGCGNGCNGACAGAGAGCA |
| | ACAAGAAGNNCCNGCCANNCCAGCAGNNNGGCCGGGANANCGCCGANACCACAGA |
| | CGCCGNNAGAGANCCCCAGACACNGGAAANCCNGGACANCACCCCNNGCAGCNNCG |
| | GCGGAGNGNCNGNGANCACCCCNGGCACCAACACCAGCAANCAGGNGGCAGNGCN |
| | GNACCAGGACGNGAACNGNACCGAAGNGCCCGNGGCCANNCACGCCGANCAGCNG |
| | ACACCNACANGGCGGGNGNACNCCACCGGCAGCAANGNGNNNCAGACCAGAGCCG |
| | GCNGNCNGANCGGAGCCGAGCACGNGAACAANAGCNACGAGNGCGACANCCCCAN |
| | CGGCGCNGGAANCNGCGCCAGCNACCAGACACAGACAAACAGCCCNCGGAGAGCCA |
| | GAAGCGNGGCCAGCCAGAGCANCANNGCCNACACAANGNCNCNGGGCGCCGAGAA |
| | CAGCGNGGCCNACNCCAACAACNCNANCGCNANCCCCACCAACNNCACCANCAGCG |
| | NGACCACAGAGANCCNGCCNGNGNCCANGACCAAGACCAGCGNGGACNGCACCANG |
| | NACANCNGCGGCGANNCCACCGAGNGCNCCAACCNGCNGCNGCAGNACGGCAGCNN |
| | CNGCACCCAGCNGAANAGAGCCCNGACAGGGANCGCCGNGGAACAGGACAAGAAC |
| | ACCCAAGAGGNGNNCGCCCAAGNGAAGCAGANCNACAAGACCCCNCCNANCAAGG |
| | ACNNCGGCGGCNNCAANNNCAGCCAGANNCNGCCCGANCCNAGCAAGCCCAGCAAG |
| | CGGAGCNNCANCGAGGACCNGCNGNNCAACAAAGNGACACNGGCCGACGCCGGCN |
| | NCANCAAGCAGNANGGCGANNGNCNGGGCGACANNGCCGCCAGGGANCNGANNNG |
| | CGCCCAGAAGNNNAACGGACNGACAGNGCNGCCNCCNCNGCNGACCGANGAGANG |
| | ANCGCCCAGNACACANCNGCCCNGCNGGCCGGCACAANCACAAGCGGCNGGACANN |
| | NGGAGCAGGCGCCGCNCNGCAGANCCCCNNNGCNANGCAGANGGCCNACCGGNNCA |
| | ACGGCANCGGAGNGACCCAGAANGNGCNGNACGAGAACCAGAAGCNGANCGCCAA |
| | CCAGNNCAACAGCGCCANCGGCAAGANCCAGGACAGCCNGAGCAGCACAGCAAGCG |
| | CCCNGGGAAAGCNGCAGGACGNGGNCAACCAGAANGCCCAGGCACNGAACACCCNG |
| | GNCAAGCAGCNGNCCNCCAACNNCGGCGCCANCAGCNCNGNGCNGAACGANANCCN |
| | GAGCAGACNGGACCCNCCNGAGGCCGAGGNGCAGANCGACAGACNGANCACAGGC |
| | AGACNGCAGAGCCNCCAGACANACGNGACCCAGCAGCNGANCAGAGCCGCCGAGAN |
| | NAGAGCCNCNGCCAANCNGGCCGCCACCAAGANGNCNGAGNGNGNGCNGGGCCAG |
| | AGCAAGAGAGNGGACNNNNGCGGCAAGGGCNACCACCNGANGAGCNNCCCNCAGN |
| | CNGCCCCNCACGGCGNGGNGNNNCNGCACGNGACANANGNGCCCGCNCAAGAGAA |
| | GAANNNCACCACCGCNCCAGCCANCNGCCACGACGGCAAAGCCCACNNNCCNAGAG |
| | AAGGCGNGNNCGNGNCCAACGGCACCCANNGGNNCGNGACACAGCGGAACNNCNA |
| | CGAGCCCCAGANCANCACCACCGACAACACCNNCGNGNCNGGCAACNGCGACGNCG |
| | NGANCGGCANNGNGAACAANACCGNGNACGACCCNCNGCAGCCCGAGCNGGACAG |
| | CNNCAAAGAGGAACNGGACAAGNACNNNAAGAACCACACAAGCCCCGACGNGGAC |
| | CNGGGCGANANCAGCGGAANCAANGCCAGCGNCGNGAACANCCAGAAAGAGANCG |
| | ACCGGCNGAACGAGGNGGCCAAGAANCNGAACGAGAGCCNGANCGACCNGCAAGA |
| | ACNGGGGAAGNACGAGCAGNACANCAAGNGGCCCNGGNACANCNGGCNGGGCNNN |
| | ANCGCCGGACNGANNGCCANCGNGANGGNCACAANCANGCNGNGNNGCANGACCA |
| | GCNGCNGNAGCNGCCNGAAGGGCNGNNGNAGCNGNGGCAGCNGCNGCAAGNNCGA |
| | CGAGGACGANNCNGAGCCCGNGCNGAAGGGCGNGAAACNGCACNACACANGANGA |
| | CNCGAGCNGGNACNGCANGCACGCAANGCNAGCNGCCCCNNNCCCGNCCNGGGNAC |
| | CCCGAGNCNCCCCCGACCNCGGGNCCCAGGNANGCNCCCACCNCCACCNGCCCCAC |
| | NCACCACCNCNGCNAGNNCCAGACACCNCCCAAGCACGCAGCAANGCAGCNCAAAA |
| | CGCNNAGCCNAGCCACACCCCCACGGGAAACAGCAGNGANNAACCNNNAGCAANAA |
| | ACGAAAGNNNAACNAAGCNANACNAACCCCAGGGNNGGNCAANNNCGNGCCAGCC |
| | ACACCCNGGAGCNAGCAAAAAAAAAAAAAAAAAAAAAAAAAAAAAAGCANANGAC |
| | NAAAAAAAAAAAAAAAAAAAAAAAAAAAAAAAAAAAAAAAAAAAAAAAAAAAAA |
| | AAAAAAAAAAAAAAAAA |

In this sequence, N is 1-methylpseudouridine.

The mRNA sequence of SEQ ID NO: 1 above is the mRNA sequence of SEQ ID NO: 2 below in which all uridine (U) bases are modified to 1-methylpseudouridine.

TABLE 4

| SEQ ID NO | RNA sequence |
|---|---|
| 2 | GAGAAUAAACUAGUAUUCUUCUGGUCCCCACAGACUCAGAGAGAACCCGCCACCA |
| | UGUUCGUGUUCCUGGUGCUGCUGCCUCUGGUGUCCAGCCAGUGUGUGAACCUGAC |
| | CACCAGAACACAGCUGCCUCCAGCCUACACCAACAGCUUUACCAGAGGCGUGUACU |
| | ACCCCGACAAGGUGUUCAGAUCCAGCGUGCUGCACUCUACCCAGGACCUGUUCCUG |
| | CCUUUCUUCAGCAACGUGACCUGGUUCCACGCCAUCCACGUGUCCGGCACCAAUGG |
| | CACCAAGAGAUUCGACAACCCCGUGCUGCCCUUCAACGACGGGGUGUACUUUGCCA |
| | GCACCGAGAAGUCCAACAUCAUCAGAGGCUGGAUCUUCGGCACCACACUGGACAGC |
| | AAGACCCAGAGCCUGCUGAUCGUGAACAACGCCACCAACGUGGUCAUCAAAGUGU |
| | GCGAGUUCCAGUUCUGCAACGACCCCUUCCUGGGCGUCUACUACCACAAGAACAAC |
| | AAGAGCUGGAUGGAAAGCGAGUUCCGGGUGUACAGCAGCGCCAACAACUGCACCU |
| | UCGAGUACGUGUCCCAGCCUUUCCUGAUGGACCUGGAAGGCAAGCAGGGCAACUU |
| | CAAGAACCUGCGCGAGUUCGUGUUUAAGAACAUCGACGGCUACUUCAAGAUCUAC |
| | AGCAAGCACACCCCUAUCAACCUCGUGCGGGAUCUGCCUCAGGGCUUCUCUGCUCU |
| | GGAACCCCUGGUGGAUCUGCCCAUCGGCAUCAACAUCACCCGGUUUCAGACACUGC |
| | UGGCCCUGCACAGAAGCUACCUGACACCUGGCGAUAGCAGCAGCGGAUGGACAGC |
| | UGGUGCCGCCGCUUACUAUGUGGGCUACCUGCAGCCUAGAACCUUCCUGCUGAAG |
| | UACAACGAGAACGGCACCAUCACCGACGCCGUGGAUUGUGCUCUGGAUCCUCUGA |
| | GCGAGACAAAGUGCACCCUGAAGUCCUUCACCGUGGAAAAGGGCAUCUACCAGAC |
| | CAGCAACUUCCGGGUGCAGCCCACCGAAUCCAUCGUGCGGUUCCCCAAUAUCACCA |
| | AUCUGUGCCCCUUCGGCGAGGUGUUCAAUGCCACCAGAUUCGCCUCUGUGUACGCC |
| | UGGAACCGGAAGCGGAUCAGCAAUUGCGUGGCCGACUACUCCGUGCUGUACAACU |
| | CCGCCAGCUUCAGCACCUUCAAGUGCUACGGCGUGUCCCCUACCAAGCUGAACGAC |
| | CUGUGCUUCACAAACGUGUACGCCGACAGCUUCGUGAUCCGGGGAGAUGAAGUGC |
| | GGCAGAUUGCCCCUGGACAGACAGGCAAGAUCGCCGACUACAACUACAAGCUGCCC |
| | GACGACUUCACCGGCUGUGUGAUUGCCUGGAACAGCAACAACCUGGACUCCAAAG |
| | UCGGCGGCAACUACAAUUACCUGUACCGGCUGUUCCGGAAGUCCAAUCUGAAGCCC |
| | UUCGAGCGGGACAUCUCCACCGAGAUCUAUCAGGCCGGCAGCACCCCUUGUAACGG |
| | CGUGGAAGGCUUCAACUGCUACUUCCCACUGCAGUCCUACGGCUUUCAGCCCACAA |
| | AUGGCGUGGGCUAUCAGCCCUACAGAGUGGUGGUGCUGAGCUUCGAACUGCUGCA |
| | UGCCCCUGCCACAGUGUGCGGCCCUAAGAAAAGCACCAAUCUCGUGAAGAACAAA |
| | UGCGUGAACUUCAACUUCAACGGCCUGACCGGCACCGGCGUGCUGACAGAGAGCA |
| | ACAAGAAGUUCCUGCCAUUCCAGCAGUUUGGCCGGGAUAUCGCCGAUACCACAGA |
| | CGCCGUUAGAGAUCCCCAGACACUGGAAAUCCUGGACAUCACCCCUUGCAGCUUCG |
| | GCGGAGUGUCUGUGAUCACCCCUGGCACCAACACCAGCAAUCAGGUGGCAGUGCU |
| | GUACCAGGACGUGAACUGUACCGAAGUGCCCGUGGCCAUUCACGCCGAUCAGCUG |
| | ACACCUACAUGGCGGGUGUACUCCACCGGCAGCAAUGUGUUUCAGACCAGAGCCG |
| | GCUGUCUGAUCGGAGCCGAGCACGUGAACAAUAGCUACGAGUGCGACAUCCCCAU |
| | CGGCGCUGGAAUCUGCGCCAGCUACCAGACACAGACAAACAGCCCUCGGAGAGCCA |
| | GAAGCGUGGCCAGCCAGAGCAUCAUUGCCUACACAAUGUCUCUGGGCGCCGAGAA |
| | CAGCGUGGCCUACUCCAACAACUCUAUCGCUAUCCCCACCAACUUCACCAUCAGCG |
| | UGACCACAGAGAUCCUGCCUGUGUCCAUGACCAAGACCAGCGUGGACUGCACCAUG |
| | UACAUCUGCGGCGAUUCCACCGAGUGCUCCAACCUGCUGCUGCAGUACGGCAGCUU |
| | CUGCACCCAGCUGAAUAGAGCCCUGACAGGGAUCGCCGUGGAACAGGACAAGAAC |
| | ACCCAAGAGGUGUUCGCCCAAGUGAAGCAGAUCUACAAGACCCCUCCUAUCAAGG |
| | ACUUCGGCGGCUUCAAUUUCAGCCAGAUUCUGCCCGAUCCUAGCAAGCCCAGCAAG |
| | CGGAGCUUCAUCGAGGACCUGCUGUUCAACAAAGUGACACUGGCCGACGCCGGCU |
| | UCAUCAAGCAGUAUGGCGAUUGUCUGGGCGACAUUGCCGCCAGGGAUCUGAUUUG |
| | CGCCCAGAAGUUUAACGGACUGACAGUGCUGCCUCCUCUGCUGACCGAUGAGAUG |
| | AUCGCCCAGUACACAUCUGCCCUGCUGGCCGGCACAAUCACAAGCGGCUGGACAUU |
| | UGGAGCAGGCGCCGCUCUGCAGAUCCCCUUUGCUAUGCAGAUGGCCUACCGGUUCA |
| | ACGGCAUCGGAGUGACCCAGAAUGUGCUGUACGAGAACCAGAAGCUGAUCGCCAA |
| | CCAGUUCAACAGCGCCAUCGGCAAGAUCCAGGACAGCCUGAGCAGCACAGCAAGCG |
| | CCCUGGGAAAGCUGCAGGACGUGGUCAACCAGAAUGCCCAGGCACUGAACACCCUG |
| | GUCAAGCAGCUGUCCUCCAACUUCGGCGCCAUCAGCUCUGUGCUGAACGAUAUCCU |
| | GAGCAGACUGGACCCUCCUGAGGCCGAGGUGCAGAUCGACAGACUGAUCACAGGC |
| | AGACUGCAGAGCCUCCAGACAUACGUGACCCAGCAGCUGAUCAGAGCCGCCGAGAU |
| | UAGAGCCUCUGCCAAUCUGGCCGCCACCAAGAUGUCUGAGUGUGUGCUGGGCCAG |
| | AGCAAGAGAGUGGACUUUUGCGGCAAGGGCUACCACCUGAUGAGCUUCCCUCAGU |
| | CUGCCCCUCACGGCGUGGUGUUUCUGCACGUGACAUAUGUGCCCGCUCAAGAGAA |
| | GAAUUUCACCACCGCUCCAGCCAUCUGCCACGACGGCAAAGCCCACUUUCCUAGAG |
| | AAGGCGUGUUCGUGUCCAACGGCACCCAUUGGUUCGUGACACAGCGGAACUUCUA |
| | CGAGCCCCAGAUCAUCACCACCGACAACACCUUCGUGUCUGGCAACUGCGACGUCG |
| | UGAUCGGCAUUGUGAACAAUACCGUGUACGACCCUCUGCAGCCCGAGCUGGACAG |
| | CUUCAAAGAGGAACUGGACAAGUACUUUAAGAACCACACAAGCCCCGACGUGGAC |
| | CUGGGCGAUAUCAGCGGAAUCAAUGCCAGCGUCGUGAACAUCCAGAAAGAGAUCG |
| | ACCGGCUGAACGAGGUGGCCAAGAAUCUGAACGAGAGCCUGAUCGACCUGCAAGA |
| | ACUGGGGAAGUACGAGCAGUACAUCAAGUGGCCCUGGUACAUCUGGCUGGGCUUU |
| | AUCGCCGGACUGAUUGCCAUCGUGAUGGUCACAAUCAUGCUGUGUUGCAUGACCA |
| | GCUGCUGUAGCUGCCUGAAGGGCUGUUGUAGCUGUGGCAGCUGCUGCAAGUUCGA |
| | CGAGGACGAUUCUGAGCCCGUGCUGAAGGGCGUGAAACUGCACUACACAUGAUGA |
| | CUCGAGCUGGUACUGCAUGCACGCAAUGCUAGCUGCCCCUUUCCCGUCCUGGGUAC |
| | CCCGAGUCUCCCCCGACCUCGGGUCCCAGGUAUGCUCCCACCUCCACCUGCCCCAC |
| | UCACCACCUCUGCUAGUUCCAGACACCUCCCAAGCACGCAGCAAUGCAGCUCAAAA |
| | CGCUUAGCCUAGCCACACCCCCACGGGAAACAGCAGUGAUUAACCUUUAGCAAUAA |
| | ACGAAAGUUUAACUAAGCUAUACUAACCCCAGGGUUGGUCAAUUUCGUGCCAGCC |
| | ACACCCUGGAGCUAGCAAAAAAAAAAAAAAAAAAAAAAAAAAAAAAGCAUAUGAC |
| | UAAAAAAAAAAAAAAAAAAAAAAAAAAAAAAAAAAAAAAAAAAAAAAAAAAAAA |
| | AAAAAAAAAAAAAAAAA |

### TEST EXAMPLE 1

### Confirmation of In Vivo Immune Activity

The in vivo immune activity was confirmed as follows for any one of the nine polymers (4C, 6B, 7B, 7D, 8B, 8C, 8D, 8E, 9D) with excellent mRNA expression efficiency prepared in Example 1 and PNP (polymer nanoparticle) comprising Covid-19 spike protein mRNA.

The in vivo immune activity experiment process is as follows. Each complex was injected intramuscularly into the gastrocnemius muscle of six mice (Week 0). Two weeks after the injection, three out of the six mice were randomly selected, and the serum was isolated to evaluate antibody formation through an enzyme-linked immunosorbent assay (ELISA), and the virus neutralizing ability of the antibody was evaluated through a plaque reduction neutralization test (PRNT). In addition, the spleen was extracted, and enzyme-linked immunoSpot (ELISpot) for interferon gamma (Interferon-g) was performed on the spleen cells to evaluate T cell immune activity.

ELISA was performed as follows. First, recombinant human coronavirus SARS-CoV-2 spike glycoprotein S1 was coated on the plate using ELISA coating buffer, and then the serum isolated from the mouse was diluted at a ratio of 1:40 to 1:2, and the reaction was performed in each well. Thereafter, HRP-labeled secondary antibodies were added to perform the reaction, and TMB solution and stop solution were added to stop the reaction. Finally, the absorbance at a wavelength of 450 nm was confirmed using a multi-well plate reader, and the results are shown in Table 5 below.

TABLE 5

| polymer | Category number | Absorbance |
|---|---|---|
| 1 | 4C | 1.09 |
| 3 | 7B | 1.03 |
| 6 | 8C | 1.07 |
| 7 | 8D | 2.98 |
| 8 | 8E | 1.22 |
| 9 | 9D | 1.68 |
| 10 | mRNA | 1 |

As shown in Table 5 above, 8D was confirmed to have the highest antibody formation, and 9D and 8E were confirmed to have the next highest antibody formation.

PRNT was diluted with various amounts of the obtained serum, reacted with 50 PFU of the corona-19 virus per well at 37° C. for 1 hour, and inoculated onto vero cells, and the cells were cultured for 3 days. Thereafter, the number of plaques formed was counted by staining the cells with crystal violet, and the serum dilution factor that reduced the number of plaques by 50% was calculated, and the results are shown in Table 6 below.

TABLE 6

| | | PRNT ND₅₀ |
|---|---|---|
| polymer | Category number | (neutralization Dose 50) |
| 1 | 4C | 38 |
| 2 | 6B | 30.67 |
| 3 | 7B | 64.33 |
| 4 | 7D | 43.33 |
| 5 | 8B | 93.33 |
| 6 | 8C | 29.67 |
| 7 | 8D | 26 |
| 9 | 9D | 29.33 |
| | PBS | 5 |
| | mRNA | 24.67 |

As shown in Table 6 above, it was confirmed that 8B, 7B, 7D, 4C, 6B, 8C, and 8D carriers showed high ND₅₀ compared to free-mRNA (mRNA without carrier).

In order to measure the generated IFN-g levels, 5*10⁵ cells and 2.5*10⁵ cells were seeded in each well of the obtained spleen cells by group, and stimulation was performed using SARS-COV-2 spike glycoprotein. Thereafter, the points generated through the ELISpot reader were counted, and the ELISpot results are shown in Table 7 below.

TABLE 7

| polymer | Category number | Spot forming units (SFU) |
|---|---|---|
| 7 | 8D | 645.06 |
| | PBS | 10.96 |
| | mRNA | 441.42 |

As shown in Table 7 above, it was confirmed that when a polymer carrier such as 8D was used, higher IFN-g was produced than mRNA without a carrier. Therefore, it can be seen that the polymer-based mRNA carrier of the present invention functions as an mRNA carrier that can induce excellent immune activity against viruses.

1. A vaccine composition for SARS-COV-2 comprising:
a SARS-COV-2 spike glycoprotein mRNA, and
a polymer comprising a repeating unit of Formula 1 below: 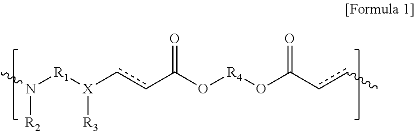
in the formula,
R1 is a bond or ethylene,
R2 and R3 are each independently (i) hydrogen, (ii) C2-5 alkyl that may be substituted with hydroxy, or (iii) N-methylpiperidine,
X is a nitrogen atom or a bond, and when X is a bond, said R3 is absent, and
R4 is a linear or methyl-substituted C3-12 alkylene, and 1 to 5 carbon atoms of said alkylene may be replaced with oxygen.

2. The vaccine composition according to claim 1, wherein the polymer comprises a repeating unit selected from the group consisting of repeating units having structures set forth in the table below

| Formula | Structure |
|---|---|
| 2 | 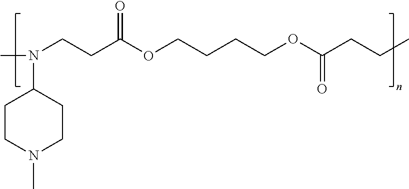 |
| 3 | 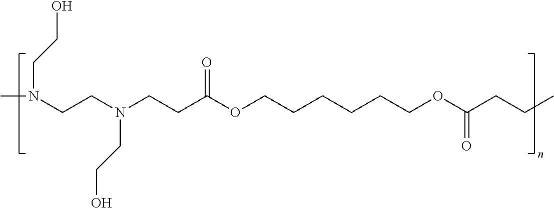 |
| 4 | 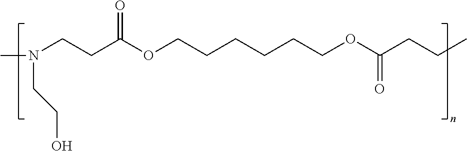 |
| 5 | 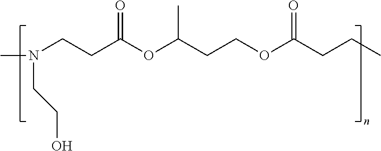 |
| 6 | 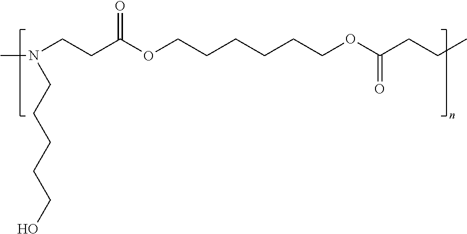 |
| 7 | 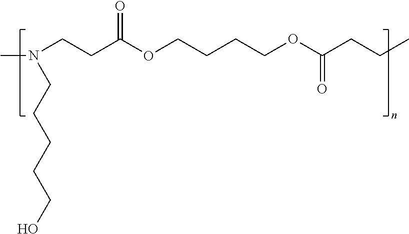 |
| 8 | 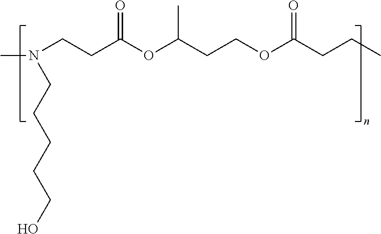 |
| 9 | 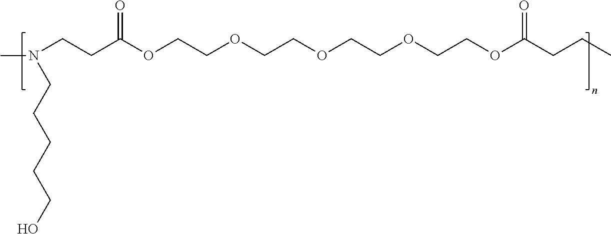 |
| 10 | 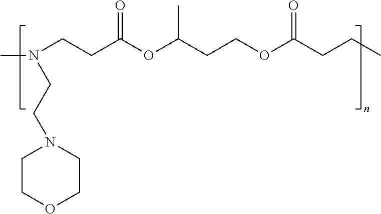 |

wherein in the formulas, n is an integer from 1 to 500.

3. The vaccine composition according to claim 1, wherein the polymer comprises a repeating unit of Formula 8, 9, or 10 below

| Formula | Structure |
|---|---|
| 8 | 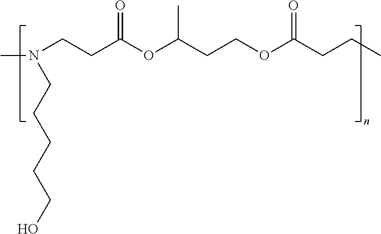 |
| 9 | 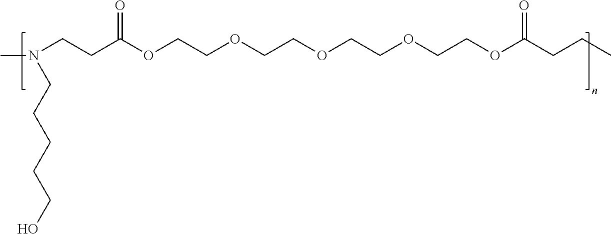 |
| 10 | 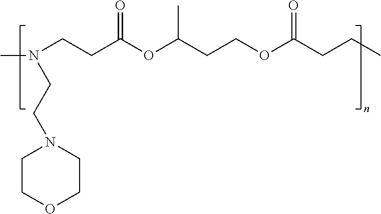 |

wherein in the formulas, n is an integer from 1 to 500.

4. The vaccine composition according to claim 1, wherein the polymer comprises a repeating unit of Formula 8 below 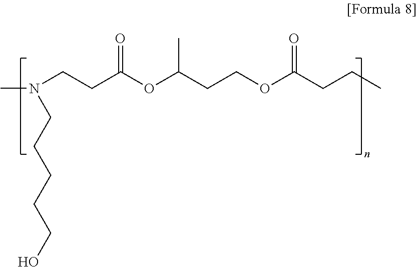
wherein in the formula, n is an integer from 1 to 500.

5. The vaccine composition according to claim 1, wherein the polymer comprises a repeating unit of Formula 9 below 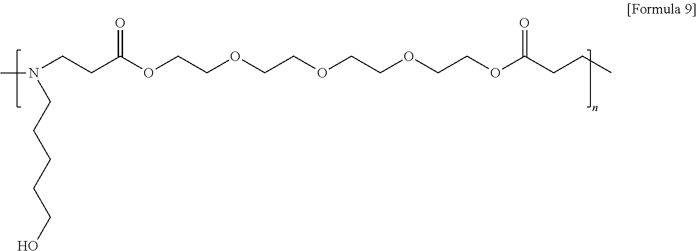
wherein in the formula, n is an integer from 1 to 500.

6. The vaccine composition according to claim 1, wherein the polymer comprises a repeating unit of Formula 10 below 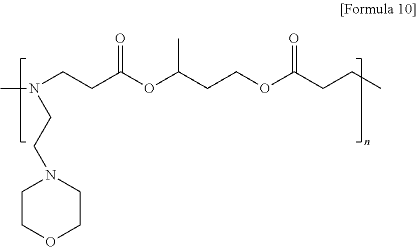
wherein in the formula, n is an integer from 1 to 500.

7. The vaccine composition according to claim 1, wherein the vaccine composition may further comprise an immune enhancer.

8. The vaccine composition according to claim 2, wherein the vaccine composition may further comprise an immune enhancer.

9. The vaccine composition according to claim 3, wherein the vaccine composition may further comprise an immune enhancer.

10. The vaccine composition according to claim 4, wherein the vaccine composition may further comprise an immune enhancer.

11. The vaccine composition according to claim 5, wherein the vaccine composition may further comprise an immune enhancer.

12. The vaccine composition according to claim 6, wherein the vaccine composition may further comprise an immune enhancer.

13. A method of inducing immune response against SARS-COV-2 comprising:
administering an effective amount of the vaccine composition according to claim 1 to a subject in need thereof.
